(19) United States
(12) Patent Application Publication
(10) Pub. No.: US 2025/0195907 A1
(43) Pub. Date: Jun. 19, 2025

(54) Wearable Therapeutic Devices and Associated Systems

(71) Applicant: Sarah Segal, Brooklyn, NY (US)

(72) Inventor: Sarah Segal, Brooklyn, NY (US)

(21) Appl. No.: 18/544,244

(22) Filed: Dec. 18, 2023

(51) Int. Cl. A61N 5/06; A41D 1/00

(52) U.S. Cl. CPC A61N 5/06; A41D 1/005; A61N 2005/0645; A61N 2005/0652

(57) ABSTRACT

The present disclosure provides generally for wearable therapeutic devices and associated systems to allow users to target specific areas of the body in need of therapeutic relief using a convenient and effective device. According to some embodiments, a wearable therapeutic device may comprise at least one light source, wherein the at least one light source may be configured to provide at least one therapeutic effect to the user. In some aspects, the light source may comprise at least one laser, wherein the laser may be configured to provide low level laser therapy (LLLT). In some aspects, the light source may comprise an LED (light-emitting diode). A wearable therapeutic device may be integrated into any article of clothing, allowing users to target specific areas in need of treatment while also allowing the users to continue with their daily routines.

### BACKGROUND

Lasers are devices that emit beams of coherent light in a process called optical amplification. A laser is unique from other light sources as it emits a coherent light, a light that has an identical waveform and frequency. The term being an acronym for light amplification by stimulated emission of radiation. Whereas the early iterations of laser technology were not suitable for practical applications, in the modern era they are used extensively.

There are five components to a typical laser: (1) a gain medium capable of sustaining stimulated emission; (2) a laser pumping energy source; (3) a high reflector; (4) an output coupler; and (5) a laser beam output. Together, the gain medium and the resonator work to power the laser and determine the beam's wavelength.

Lasers are used in a variety of technologies such as Bluetooth®, laser surgery, holographic imaging, fiber-optic communication, traffic enforcement, the military, and much more. In terms of medical applications, lasers have become very popular, being used in a variety of surgeries and treatments. For example, laser technology is being used to treat cancer by attacking and eradicating tumors and precancerous growths. Laser treatment methods are advantageous in that they are more precise than typical surgical techniques while also doing less damage to the surrounding area.

One treatment method that utilizes laser technology is low level laser therapy (LLLT), which is the practice of using low level lasers to treat musculoskeletal conditions, acting to accelerate connective tissue repair while also working as an anti-inflammatory agent. These techniques can reduce inflammation in the matter of hours for fast pain relief, and they are often used on people suffering from osteoarthritis, rheumatoid arthritis, tennis elbow, disc herniation, tendonitis, neuropathic pain, and other musculoskeletal conditions. More recently, LLLT devices have been developed for at-home use for more general applications on wounds, ulcers, and other relevant sources of pain.

While these developments in laser technology have greatly improved the quality of life of individuals suffering from both chronic and acute conditions and pain, there are opportunities in these advancements that have yet to be addressed. Current at-home LLLT technologies may require a user to stop what they are doing multiple times a day to administer treatment for a considerable duration. This is impractical for many who lead busy lives and are not home or at another convenient location at which to administer the treatment. LLLT lasers that can adapt to an individual's everyday lifestyle, allowing for easier and more accommodating usage, may comprise an opportunity for improvement.

### SUMMARY OF THE DISCLOSURE

What is needed are wearable therapeutic devices and associated systems to allow users to target specific areas on the body in need of therapeutic relief using a convenient and effective device. Such a device may allow users to benefit from the effects of the wearable therapeutic device at any time of day by providing a simple and accessible alternative to other light therapies. A wearable therapeutic device may be integrated into any article of clothing, allowing a user to target specific areas in need of treatment while also allowing the user to continue with daily routines without significant interruption.

The present disclosure provides generally for wearable therapeutic devices and associated systems. According to some embodiments, a wearable therapeutic device may comprise at least one light source, wherein the at least one light source may be configured to provide at least one therapeutic effect to a user. In some aspects, the at least one light source may comprise at least one laser, wherein the laser may be configured to provide low level laser therapy (LLLT). In some embodiments, the at least one light source may comprise at least one collimated and naturally diverging laser. In some aspects, the at least one light source may comprise an LED (light-emitting diode) light. In some aspects, the wearable therapeutic device may comprise at least one power source, wherein the at least one power source may be configured to supply power to the at least one light source. In some implementations, the wearable therapeutic device may comprise at least one receptacle, wherein the at least one receptacle may be configured to secure the at least one light source and the at least one power source to one or more articles of clothing.

In some embodiments, the wearable therapeutic device of the present disclosure may comprise at least one lens, wherein the at least one light source may emit light through the at least one lens. In some aspects, the wearable therapeutic device may comprise at least one housing, wherein the at least one housing may comprise the at least one light source and the at least one lens. In some embodiments, the wearable therapeutic device may comprise at least one circuit board, wherein the at least one circuit board may connect the at least one light source and the at least one power source. In some aspects, the wearable therapeutic device may comprise one or more electrical connections, wherein the one or more electrical connection may facilitate at least one at least partial electrical connection between the at least one light source and the at least one power source through the at least one circuit board. In some aspects, the one or more electrical connections may comprise electrical wires, threads, or any other mechanism that may facilitate an electrical connection. In some implementations, the wearable therapeutic device may comprise a first at least one connection mechanism, wherein the first at least one connection mechanism may be configured to complete an electrical circuit between the at least one power source and the at least one light source. In some aspects, the connection mechanism may comprise one or more magnets, one or more clasps, or one or more hook and loop mechanisms, as non-limiting examples. In some implementations, the wearable therapeutic device may comprise a second at least one connection mechanism, wherein the second at least one connection mechanism may be configured to connect the wearable therapeutic device to at least one portion of the at least one receptacle.

In some embodiments, the wearable therapeutic device may be configured to be at least temporarily integrated with at least one portion of one or more various articles of clothing. In some aspects, the wearable therapeutic device may be securely or removably attached to an article of clothing at a location proximate to a portion of a user's body that may benefit from exposure light emitted from the light source. By way of example and not limitation, if a user is experiencing shoulder pain, the user may place the wearable therapeutic device at or near the location of the pain, such that the area may receive at least one therapeutic effect from the light source.

In some aspects, the wearable therapeutic device may be configured to move in at least one direction within or upon an article of clothing. In some non-limiting exemplary embodiments, movement of the wearable therapeutic device may be facilitated remotely. By way of example and not limitation, a user may indicate an area on the body in need of laser light therapy using at least one computing device, and the wearable therapeutic device maybe be configured to move to the indicated position, where it may be most advantageous to achieve the desired therapeutic effect.

In some implementations, the wearable therapeutic device may comprise at least one transportation apparatus. In some embodiments, the transportation mechanism may facilitate movement of the wearable therapeutic device to various areas of the body of a user in order to provide a therapeutic effect to different areas. In some aspects, the transportation apparatus may comprise at least one of: a track, a belt system, or a pulley system, as non-limiting examples. In some embodiments, the wearable therapeutic device may comprise at least one sensor, wherein the at least one sensor may be configured to sense or detect at least one datum of information related to the use of the wearable therapeutic device. In some aspects, the wearable therapeutic device may comprise at least one transmitting device, wherein the at least one transmitting device may be configured to transmit the at least one datum to at least one computing device. In some aspects, the at least one computing device may be configured to perform or execute one or more analytical functions on the at least one datum.

### BRIEF DESCRIPTION OF THE DRAWINGS

The accompanying drawings that are incorporated in and constitute a part of this specification illustrate several embodiments of the disclosure and, together with the description, serve to explain the principles of the disclosure:

FIG. 1A illustrates an exemplary wearable therapeutic device, according to some embodiments of the present disclosure.

FIG. 1B illustrates an exemplary wearable therapeutic device, according to some embodiments of the present disclosure.

FIG. 1C illustrates an exemplary wearable therapeutic device, according to some embodiments of the present disclosure.

FIG. 2A illustrates an article of clothing as a shirt comprising an exemplary wearable therapeutic device, according to some embodiments of the present disclosure.

FIG. 2B illustrates an article of clothing as a shirt comprising an exemplary wearable therapeutic device, according to some embodiments of the present disclosure.

FIG. 2C illustrates an article of clothing as a shirt comprising an exemplary wearable therapeutic device, according to some embodiments of the present disclosure.

FIG. 2D illustrates an article of clothing as a shirt comprising an exemplary wearable therapeutic device, according to some embodiments of the present disclosure.

FIG. 2E illustrates an article of clothing as a shirt comprising an exemplary wearable therapeutic device, according to some embodiments of the present disclosure.

FIG. 3A illustrates an article of clothing as a shirt comprising an exemplary wearable therapeutic device, according to some embodiments of the present disclosure.

FIG. 3B illustrates an article of clothing as a shirt comprising an exemplary wearable therapeutic device, according to some embodiments of the present disclosure.

FIG. 4A illustrates an article of clothing as a jacket comprising an exemplary wearable therapeutic device, according to some embodiments of the present disclosure.

FIG. 4B illustrates an article of clothing as a jacket comprising an exemplary wearable therapeutic device, according to some embodiments of the present disclosure.

FIG. 5 illustrates an article of clothing as a pair of glasses comprising an exemplary wearable therapeutic device, according to some embodiments of the present disclosure.

FIG. 6 illustrates an article of clothing comprising an exemplary wearable therapeutic device, according to some embodiments of the present disclosure.

FIG. 7 illustrates an exemplary wearable therapeutic device, according to some embodiments of the present disclosure.

### DETAILED DESCRIPTION

The present disclosure provides generally for wearable therapeutic devices and associated systems to allow users to target specific areas in need of therapeutic relief using a convenient and effective device. According to some embodiments, a wearable therapeutic device may comprise at least one light source, wherein the light source may be configured to provide at least one therapeutic effect to a user. In some aspects, the light source may comprise a laser, wherein the laser may be configured to provide low level laser therapy (LLLT). In some embodiments, the light source may comprise at least one collimated and naturally diverging laser. In some aspects, the light source may comprise an LED (light-emitting diode) light. In some implementations, the wearable therapeutic device may facilitate the ability of a user to benefit from one or more therapeutic effects of the wearable therapeutic device at any time of day by providing a simple and accessible alternative to other light therapy systems. In some embodiments, the wearable therapeutic device may be configured to be at least temporarily integrated with at least one portion of any article of clothing, thereby allowing a user to receive one or more beneficial effects provided by the wearable therapeutic device while also allowing the user to continue with one or more daily routines without significant interruption.

In the following sections, detailed descriptions of examples and methods of the disclosure will be given. The descriptions of both preferred and alternative examples, though thorough, are exemplary only, and it is understood to those skilled in the art that variations, modifications, and alterations may be apparent. It is therefore to be understood that the examples do not limit the broadness of the aspects of the underlying disclosure as defined by the claims.

### Glossary

Light source: as used herein refers to any device that may emit one or more wavelengths of light that may provide at least one therapeutic effect to a user. By way of example and not limitation, a single light source may comprise one or more wavelengths. By way of example and not limitation, a light source may comprise at least one laser. In some aspects, the laser may be configured to provide pain relief, skin condition relief, hair loss prevention or relief, emotional relief, or mental relief, as non-limiting examples. In some aspects, a light source may comprise at least one LED (light-emitting diode). In some aspects, the LED light may be configured to provide pain relief, skin condition relief, hair loss prevention or relief, emotional relief or mental relief, as non-limiting examples.
Therapeutic effect: as used herein refers to any physical, mental, or emotional relief or benefit to a user that may result from the use of at least one light source. By way of example and not limitation, a therapeutic effect may comprise pain relief, skin condition relief, hair loss prevention or relief, eye disease relief, emotional relief, or mental relief, as non-limiting examples.
Wearable therapeutic device: as used herein refers to any device that may be worn by a user that may comprise a light source. By way of example and not limitation, a wearable therapeutic device may comprise at least one LLLT light source, wherein the LLLT light source is configured to provide at least one therapeutic effect to a user, as non-limiting examples.
Article of clothing: as used herein refers to any article of clothing that may comprise at least one therapeutic device. By way of example and not limitation, an article of clothing may comprise shirts, pants, jackets, shoes, scarves, eyeglasses, hats, masks, socks, gloves as non-limiting examples.

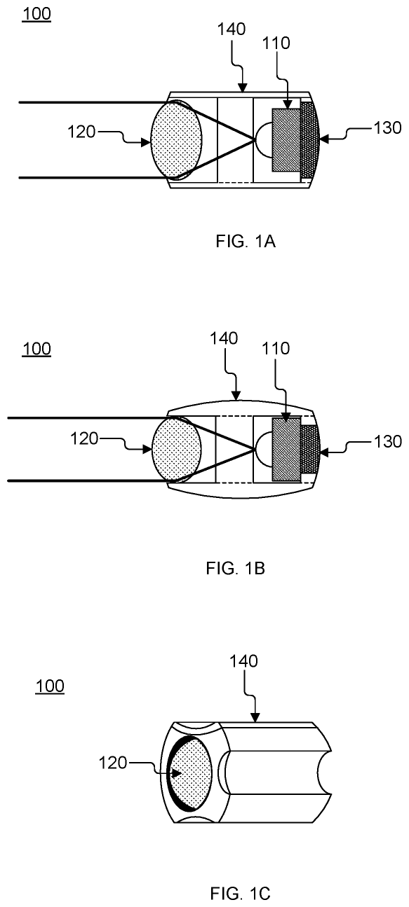

Referring now to FIGS. 1A, 1B, and 1C, exemplary wearable therapeutic devices 100, according to some embodiments of the present disclosure, are illustrated. In some aspects, the wearable therapeutic device 100 may comprise at least one light source 110, wherein the light source 110 may be configured to provide at least one therapeutic effect to a user. By way of example and not limitation, the light source 110 may be configured to provide relief for chronic or temporary pain. In some aspects, the light source 110 may comprise at least one laser, wherein the laser may be configured to provide low level laser therapy (LLLT). In some embodiments, the light source 110 may comprise at least one collimated and naturally diverging laser.

By way of example and not limitation, the light source 110 comprising a laser may be used to treat chronic or temporary pain by reducing inflammation, which may be a leading cause of pain. In some aspects, the light source 110 comprising a laser may facilitate one or more biological reactions that may help treat chronic or temporary pain, such as by increasing blood circulation, metabolic processes, or production of collagen and other proteins, as non-limiting examples. In some aspects, the light source 110 may comprise at least one LED (light-emitting diode). By way of example and not limitation, the light source 110 comprising an LED may be used to treat one or more of: pain, skin conditions, hair loss, eye conditions, emotional conditions, or mental conditions, as non-limiting examples.

In some embodiments, the wearable therapeutic device 100 may comprise at least one lens 120, wherein the light source 110 may emit light through the lens 120. In some implementations, the lens 120 may be configured to focus the light emitted by the light source 110. In some implementations, the focus of the light facilitated by the lens 120 may alter the intensity of the light emitted from the light source 110. In some aspects, the lens 120 may be configured for the natural divergence of light.

By way of example and not limitation, the light source 110 comprising a laser may be configured to emit light through the lens 120, and the lens 120 may focus the light emitted by the laser. In some aspects, the lens 120 may comprise one or more of a plurality of different sizes and shapes to provide different focal points for the light emitted from the light source 110. In some aspects, adjusting the focal point of the light emitted from the light source 110 may enable the light source 110 to produce a range of therapeutic effects. By way of example and not limitation, when the light emitted from the light source 110 through the lens 120 comprises a focal point that concentrates a greater power of the light emitted from the light source 110 upon a user's body, the user may experience greater therapeutic effects from the wearable therapeutic device 100.

In some aspects, the wearable therapeutic device 100 may comprise at least one housing 140, wherein the housing 140 may comprise the light source 110 and the lens 120. In some implementations, the light source 110 and the lens 120 may be at least partially enclosed within the housing 140. In some embodiments, the housing 140 may be shaped or otherwise configured to facilitate efficient manual relocation or movement of the wearable therapeutic device 100 by a user. By way of example and not limitation, the housing 140 may comprise four semi-circular indentations, wherein each indentation may be configured to receive at least a portion of at least one of the user's fingers such that it may be easier for the user to grip or grasp the wearable therapeutic device 100 while moving the wearable therapeutic device 100. In some aspects, this may allow the user to easily move the wearable therapeutic device 100 to different locations within or upon an article of clothing that may be proximate to different portions of the user's body, which may increase the locations at which therapeutic effects facilitated by the wearable therapeutic device 100 may be received or experienced by the user.

In some implementations, the wearable therapeutic device 100 may comprise a first at least one connection mechanism 130, wherein the first connection mechanism 130 may be configured to facilitate completion of an electrical circuit between a power source and the light source 110. In some aspects, the connection mechanism may comprise one or more magnets, one or more clasps, or one or more hook and loop mechanisms, as non-limiting examples. In some aspects, the first connection mechanism 130 may be located within or upon at least one portion of the housing 140. By way of example and not limitation, the light source 110 may be configured within or upon a portion of the housing 140 relative to the lens 120 to facilitate emission of light through the lens 120. In some aspects, the first connection mechanism 130 may be electrically connected to the light source 110 via one or more electrical connections. In some embodiments, the wearable therapeutic device 100 may comprise a second at least one connection mechanism. In some aspects, the one or more electrical connections may comprise electrical wires, threads, or any other mechanism that may facilitate an electrical connection. In some aspects, the second connection mechanism may connect to the power source via one or more electrical connections.

By way of example and not limitation, the second connection mechanism may be configured within or upon at least one portion of at least one receptacle configured to at least partially enclose one or more components of the wearable therapeutic device 100. In some implementations, the first connection mechanism 130 may connect to the second connection mechanism, thus completing the electrical circuit and powering the light source 110. In some aspects, upon connection of the first connection mechanism 130 and the second connection mechanism, electricity may flow from the power source through the electrical connection to the second connection mechanism, through the first connection mechanism, and through electrical thread to the light source 110.

In some implementations, the connection mechanism 130 may also facilitate movement and relocation of the wearable therapeutic device 100 within or upon an article of clothing. By way of example and not limitation, a user may detach the connection mechanism 130 from its corresponding second connection mechanism to move the wearable therapeutic device 100 to one or more different locations. In some aspects, this may allow a user to easily move the wearable therapeutic device 100 to be proximate to different portions of the body, which may increase the number of places at which the user may receive or experience the therapeutic effects facilitated by the wearable therapeutic device 100.

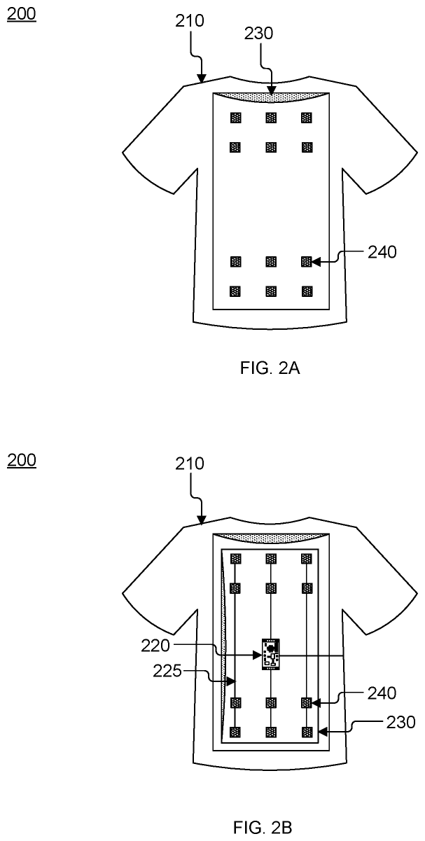

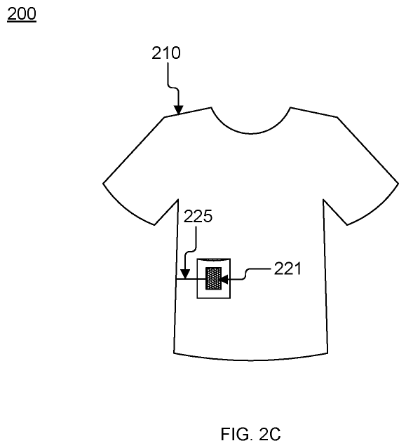

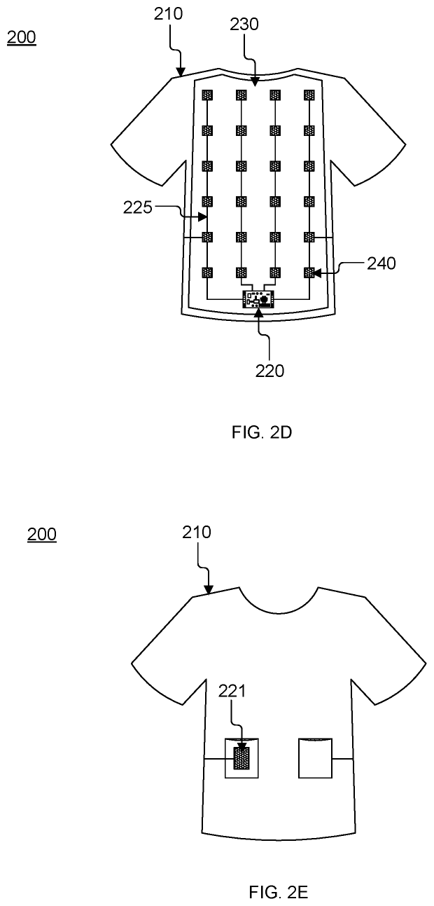

Referring now to FIGS. 2A, 2B, 2C, 2D, and 2E, articles of clothing as a shirt 210 comprising an exemplary wearable therapeutic device 200, according to some embodiments of the present disclosure, are illustrated. In some implementations, the wearable therapeutic device 200 may comprise at least one receptacle 230. In some aspects, the receptacle 230 may be configured to at least temporarily receive and at least partially enclose one or more various electrical components, such as at least one light source and at least one power source 221.

By way of example and not limitation, the receptacle 230 may be configured to at least temporarily secure a housing of the wearable therapeutic device 200 to at least one portion of one or more articles of clothing 210, wherein the housing may comprise at least one light source and at least one lens, and at least one power source 221 to one or more articles of clothing 210. In some aspects, the receptacle 230 may comprise at least one attachment mechanism configured to removably attach the receptacle 230 to any article of clothing 210, wherein the attachment mechanism may comprise at least one of: a zipper, a hook-and-loop fastener, a snap fastener, or a magnetic mechanism, as non-limiting examples.

By way of example and not limitations, the receptacle 230 may be removed from the article of clothing 210 by the user. This may allow for the user to easily remove the receptacle 230 containing the electrical components so that the user may wash the clothing 210 without the worry of damaging the electrical components. In some embodiments, the receptacle 230 may be configured to removably attach to various articles of clothing 210 in various locations. By way of example and not limitation, a user may secure the receptacle 230 proximate to areas in need of therapeutic treatment, such as a shoulder experiencing chronic pain, as a non-limiting example.

In some aspects, the wearable therapeutic device 200 may comprise at least one power source 221, wherein the power source 221 may be configured to supply power to the light source. By way of example and not limitation, the power source 221 may comprise at least one battery. In some aspects, the battery may be configured to be rechargeable. This may allow for a user to easily supply power to the light source. In some aspects, the power source 221 may be at least temporarily positioned within or upon the receptacle 230 at one or more various locations.

By way of example and not limitation, the power source 221 may be positioned in an area to facilitate a comfortable experience for a user, such as at a distance on the article of clothing 210 to counterbalance the weight of the various electrical components. For example, an article of clothing 210, such as a shirt, may comprise a receptacle 230 that allows for the power source 221 to be on the front side of the shirt and the remaining electrical components may be configured on the back side of the shirt to create a weight balance that may be comfortable to the user.

In some embodiments, the wearable therapeutic device 200 may comprise at least one circuit board 220, wherein the circuit board 220 may be configured to connect the light source and the power source 221. In some aspects, the receptacle 230 may comprise the circuit board 220, wherein the circuit board 220 may be at least partially enclosed within the receptacle 230. In some aspects, the wearable therapeutic device 200 may comprise one or more electrical connections 225, wherein the electrical connection 225 may be configured to facilitate at least one at least partial electrical connection between the light source and the power source 221 through the at least one circuit board 220.

In some aspects, the one or more electrical connections may comprise electrical wires, threads, or any other mechanism that may facilitate an electrical connection. In some implementations, the wearable therapeutic device 200 may comprise a first at least one connection mechanism 240, wherein the first connection mechanism 240 may be connected to the light source via the electrical connection 225, and wherein the first connection mechanism 240 may be configured to removably connect the housing to the receptacle 230. In some aspects, the connection mechanism may comprise one or more magnets, one or more clasps, or one or more hook and loop mechanisms, as non-limiting examples. In some aspects, the electrical connections 225 may be configured to connect a second connection mechanism 240 to the power source 221 within the receptacle 230, such that when a corresponding first connection mechanism 240 connected to a light source is attached to the second connection mechanism 240 connected to the power source 221, an electrical circuit may be completed between the light source and the power source 221. In some aspects, the second connection mechanism 240 may be configured within or upon at least one portion of the receptacle 230.

By way of example and not limitation, the second connection mechanism 240 may be positioned at a location proximate to a portion of a user's body in need of therapeutic treatment, such as a shoulder. This may allow a user to use the first connection mechanism 240 to connect the housing, with the light source, to the second connection mechanism 240 near the shoulder to facilitate a therapeutic effect, such as pain relief. In some aspects, the receptacle 230 may comprise a plurality of second connection mechanisms 240. This may allow a user to connect the housing at various locations within or upon the receptacle 230 by attaching the first connection mechanism 240 of the housing to different second connection mechanisms 240 at different locations. For example, a user who experiences pain in both their shoulder and lower back may move the housing between the two areas as needed to facilitate therapeutic treatment.

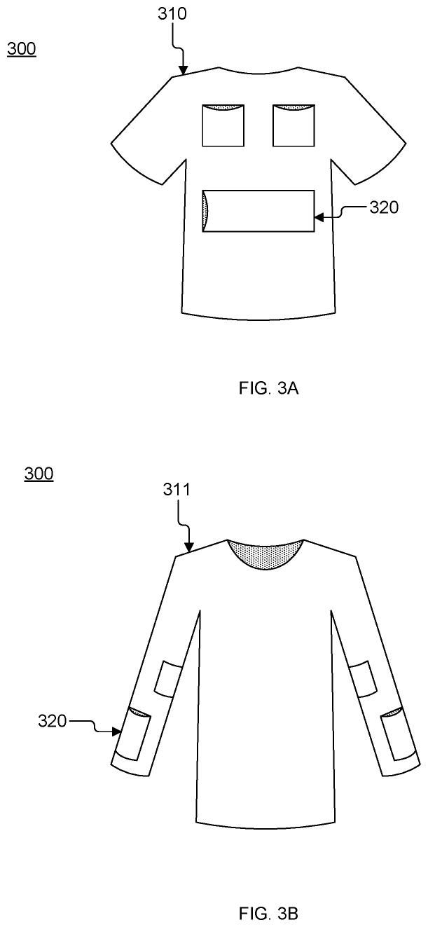

Referring now to FIGS. 3A and 3B, articles of clothing 310, 311 as a shirt comprising an exemplary wearable therapeutic device 300, according to some embodiments of the present disclosure, are illustrated. In some embodiments, the wearable therapeutic device 300 may be configured to attach to one or more various of articles of clothing 310, 311. By way of example and not limitation, the article of clothing 310 may comprise a short-sleeved shirt. By way of further example and not limitation, the article of clothing 311 may comprise a long-sleeved shirt 311. In some aspects, the wearable therapeutic device 300 may be positioned at one or more at least temporarily securable locations within or upon the article of clothing 310, 311 via at least one receptacle 320. In some embodiments, the receptacle 320 may be configured to attach to various articles of clothing 310, 311 in various locations using at least one attachment mechanism.

By way of example and not limitation, a user may at least temporarily secure the receptacle 320 proximate to one or more areas in need of therapeutic treatment, such as a shoulder experiencing chronic pain. By way of further example and not limitation, the receptacle 320 may be positioned on the wrist or elbow area of a long-sleeved shirt 311, which may be proximate to portions of a user's body that often experience joint pain or other disorders. This may allow a user to focus the light emitted from the light source in areas most in need of treatment. The receptacle 320 may allow a user to conveniently operate the wearable therapeutic device 300, which may allow a user to continue their daily routines while receiving therapeutic treatment for a variety of ailments.

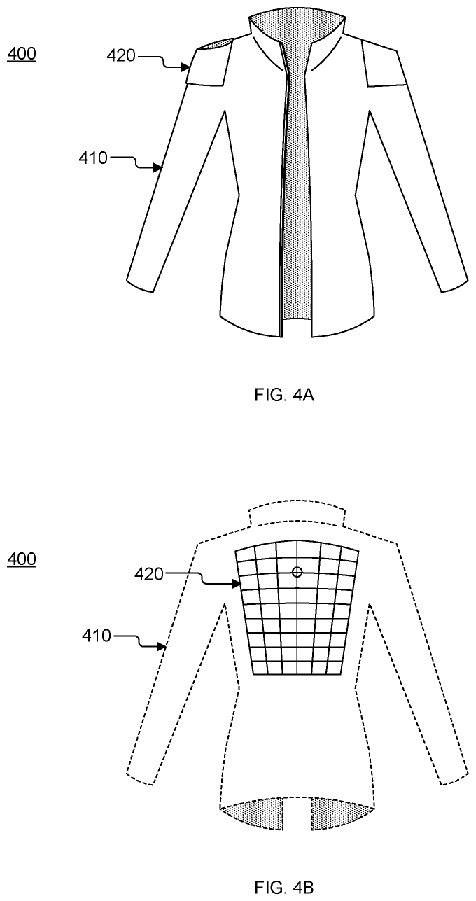

Referring now to FIGS. 4A and 4B, articles of clothing as a jacket 410 comprising an exemplary wearable therapeutic device 400, according to some embodiments of the present disclosure, are illustrated. In some embodiments, the wearable therapeutic device 400 may be configured to attach to one or more various of articles of clothing 410. By way of example and not limitation, the article of clothing 410 may comprise a sports jacket. In some aspects, the wearable therapeutic device 400 may be positioned on the sports jacket 410 via at least one receptacle 420. In some embodiments, the receptacle 420 may be configured to attach to various articles of clothing 410 in various locations using at least one an attachment mechanism.

By way of example and not limitation, a user may at least temporarily secure the receptacle 420 proximate to one or more portions of the user's body in need of therapeutic treatment, such as an upper or lower back experiencing chronic pain. By way of further example and not limitation, the receptacle 420 may be positioned on the back area of the jacket 410, which may be proximate to an area that causes pain for many users. This may allow a user to focus the light source in areas most in need of treatment, such as the upper and lower back. The receptacle 420 may allow a user to conveniently operate the wearable therapeutic device 400, which may allow a user to continue their daily routines while receiving therapeutic treatment for a variety of ailments. For example, a user may wear a sports jacket with the wearable therapeutic device 400 while exercising, which may allow a user to treat or mitigate back pain while exercising.

In some implementations, the receptacle 420 may comprise at least one transportation apparatus for the wearable therapeutic device 400. In some embodiments, the transportation mechanism may facilitate movement of the wearable therapeutic device 400 to various areas of the body of a user in order to provide a therapeutic effect to different areas. In some aspects, the transportation apparatus may comprise at least one of: a track, a grid system, a belt system, or a pulley system, as non-limiting examples.

By way of example and not limitation, this may allow a user to remotely or manually move the wearable therapeutic device 400 via the transportation apparatus to different areas of an article of clothing proximate to different portions of the body to target different areas in need of therapeutic treatment. In some non-limiting exemplary embodiments, the wearable therapeutic device 400 may comprise at least one housing, wherein the housing may comprise the light source and at least one lens, and the transportation apparatus may be configured to facilitate movement of the housing within the receptacle 420.

In some aspects, the wearable therapeutic device 400 may be configured to move automatically, such as after a period of time or upon the happening of an event, such as if a user were to interact with a switch or button integrated with a signaling device or a controller. In some aspects, the movement of the wearable therapeutic device 400 may be facilitated remotely. By way of example and not limitation, a user may indicate an area on the body in need of treatment using at least one computing device, and the wearable therapeutic device 400 may be configured to move to a position most advantageous to achieve the desired therapeutic effect as directed by the computing device. For example, a user may indicate on a computing device that they are experiencing neck pain. In some aspects, this indication may cause the computing device to instruct the wearable therapeutic device 400 to move to one or more various locations proximate to the neck to provide therapeutic treatment.

In some aspects, the wearable therapeutic device 400 may comprise at least one sensor, wherein the sensor may be configured to sense or detect information that comprises at least one datum related to the use of the wearable therapeutic device 400, wherein the wearable therapeutic device 400 may comprise at least one database or other storage medium configured to at least temporarily store sensor data.

By way of example and not limitation, the sensor may be configured to detect temperature, skin conditions such as moisture levels, or movement of the user, as non-limiting examples. For example, the sensor may detect that a user is moving or exercising an area in need of treatment. In some aspects, the wearable therapeutic device 400 may comprise at least one transmitting device or transmitter, wherein the transmitter may be configured to transmit the sensor data to at least one computing device. By way of example and not limitation, the computing device may comprise at least one of: a desktop computer, a mobile device, or a smartwatch, as non-limiting examples. In some aspects, the computing device may be configured to perform or execute one or more analytical functions on the data.

By way of example and not limitation, the computing device may analyze the movement data obtained by the sensor and activate the wearable therapeutic device 400 when a user is moving or exercising. By way of further example and not limitation, the computing device may analyze body temperature data of the user and increase or decrease the amount of time the light source may be active in a particular area based on the temperature of the user's body.

In some embodiments, the wearable therapeutic device 400 may be configured to attach to one or more various of articles of clothing. By way of example and not limitation, the article of clothing may comprise a pair of pants. In some aspects, the wearable therapeutic device may be at least temporarily secured to at least one location position on the pair of pants via at least one receptacle. In some embodiments, the receptacle may be configured to removably attach various articles of clothing in various locations.

For example, a user may secure the receptacle proximate to one or more portions of the user's body in need of therapeutic treatment, such as a knee or hip joint experiencing chronic pain. In some aspects, a user may secure the receptacle at or near the waistline of the pair of pants to provide therapeutic treatment to at least one hip joint. By way of further example and not limitation, the receptacle may be positioned at or near the knee region of a pair of pants, which may be proximate to areas of a user's body that often experience joint pain or other disorders.

By way of example and not limitation, the article of clothing may comprise a dress. In some aspects, the wearable therapeutic device 400 may be positioned on the dress via at least one receptacle. In some embodiments, the receptacle may be configured to removably attach to one or more portions of various articles of clothing in various locations. By way of example and not limitation, a user may secure the receptacle proximate to portions of the user's body in need of therapeutic treatment, such as a hip joint experiencing chronic pain. By way of further example and not limitation, the receptacle may be positioned upon or with one or more shoulder straps of the dress, which may be areas that often experience joint pain or other disorders.

By way of example and not limitation, the article of clothing may comprise at least one glove. In some aspects, the wearable therapeutic device 400 may be positioned within or upon at least one portion of the glove via at least one receptacle. By way of example and not limitation, a user may secure the receptacle proximate to portions of the user's body in need of therapeutic treatment, such as near one or more fingers experiencing symptoms of carpal tunnel syndrome.

By way of example and not limitation, the article of clothing may comprise a hat. In some aspects, the wearable therapeutic device may be positioned on the hat via at least one receptacle. By way of example and not limitation, a user may secure the receptacle proximate to one or more areas in need of therapeutic treatment, such as scalp experiencing hair loss or other skin conditions.

By way of example and not limitation, the article of clothing may comprise at least one shoe. By way of further example and not limitation, the article of clothing may comprise at least one sock. In some aspects, the wearable therapeutic device may be positioned within or upon the article of clothing via at least one receptacle. By way of example and not limitation, a user may secure the receptacle proximate to one or more areas in need of therapeutic treatment, such an ankle experiencing pain. By way of further example and not limitation, the receptacle may be positioned upon or within a sock or shoe which may comprise areas that often experience joint pain, skin disorders, or other health issues.

By way of example and not limitation, the article of clothing may comprise a scarf. By way of further example and not limitation, the article of clothing may comprise a sash. In some aspects, the wearable therapeutic device may be positioned within or upon the scarf to the sash via at least one receptacle.

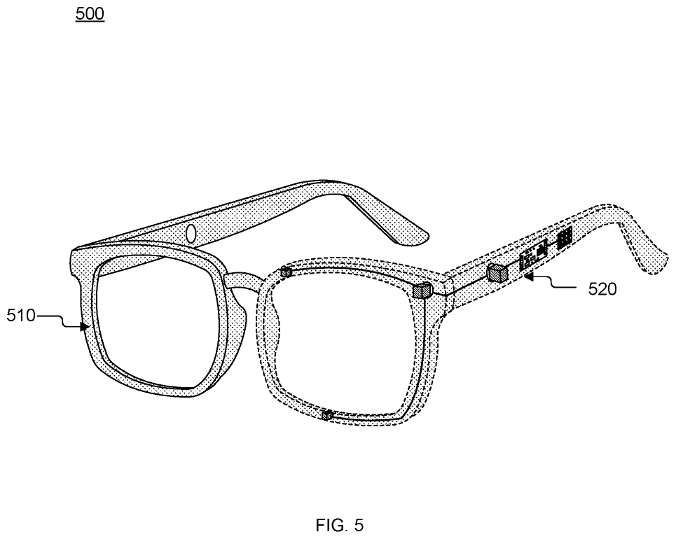

Referring now to FIG. 5, an article of clothing as a pair of glasses 510 comprising an exemplary wearable therapeutic device 500, according to some embodiments of the present disclosure, is illustrated. In some embodiments, the wearable therapeutic device 500 may be configured to attach to one or more various of articles of clothing 510. By way of example and not limitation, the article of clothing 510 may comprise a pair of glasses. In some aspects, the wearable therapeutic device 500 may be positioned on the pair of glasses 510 via at least one receptacle 520. In some embodiments, the receptacle 520 may be configured to removably attach to one or more portions of one or more various articles of clothing 510 in various locations. By way of example and not limitation, a user may secure the receptacle 520 proximate to one or more areas in need of therapeutic treatment, such an eye experiencing various eye conditions, such as glaucoma, retinopathy, or eye pain, as non-limiting examples. This may allow a user to focus the light source in areas most in need of treatment, such as, for example and not limitation, one or more locations proximate to, on, or within an eye, similar to the locations that may be targeted, for example, during repetitive transorbital alternating current stimulation. In some implementations, the receptacle 520 may allow a user to conveniently operate the wearable therapeutic device 500, which may allow the user to continue their daily routines while receiving therapeutic treatment for a variety of ailments.

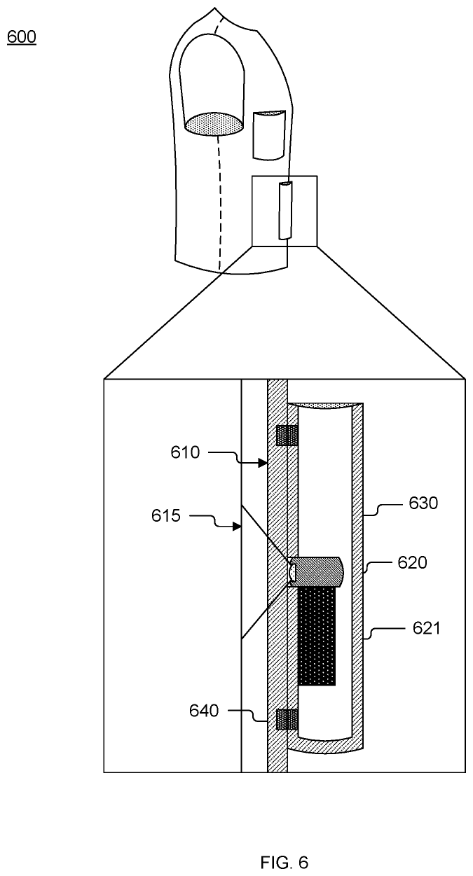

Referring now to FIG. 6, an article of clothing 610 comprising an exemplary wearable therapeutic device 600 positioned within an article of clothing worn by a user, according to some embodiments of the present disclosure, is illustrated. In some implementations, the wearable therapeutic device 600 may comprise at least one receptacle 630. By way of example and not limitation, the receptacle 630 may be configured to at least temporarily secure a housing 620 containing at least one light source and at least one lens, and at least one power source 621 to one or more portions of one or more articles of clothing 610. In some aspects, the receptacle 630 may comprise at least one attachment mechanism 640 configured to removably or securely attach the receptacle 630 to any article of clothing 610. In some aspects, the attachment mechanism 640 may comprise at least one of: a zipper, a hook-and-loop fastener, a magnetic mechanism, or a snap fastener, as non-limiting examples. This may allow for the user to remove and reattach the receptacle 630.

In some embodiments, the receptacle 630 may be configured to attach to one or more various articles of clothing in various locations. By way of example and not limitation, a user may at least temporarily secure the receptacle 630 proximate to one or more areas in need of therapeutic treatment, such as a shoulder experiencing chronic pain. By way of example and not limitation, the attachment of the receptacle 630 to an article of clothing 610 may facilitate therapeutic treatment of an ailment via the light source by securing the housing 620 close to the surface of a user's skin 615. This may allow treatment of various ailments, such as pain, hair loss, and skin conditions, as non-limiting examples. In some aspects, the light source may be configured to provide therapeutic effects to a user through one or more materials of an article of clothing 610, such as a shirt, pants, hat, gloves, scarf, dress, jacket, shoes, or socks, as non-limiting examples.

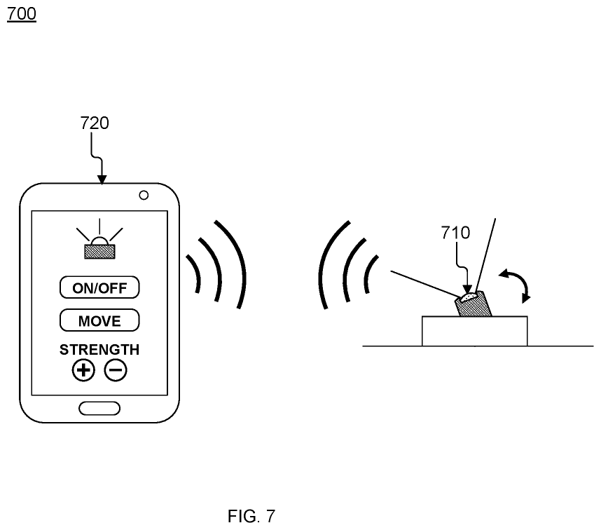

Referring now to FIG. 7, an exemplary wearable therapeutic device 700, according to some embodiments of the present disclosure, is illustrated. In some implementations, the wearable therapeutic device 700 may comprise at least one receptacle 720, wherein the receptacle 720 may comprise at least one transportation apparatus configured to facilitate movement of the wearable therapeutic device 700 in at least one direction. In some aspects, the transportation apparatus may comprise at least one of: a track, a grid system, a belt system, or a pulley system, as non-limiting examples. In some implementations, the transportation apparatus may move the wearable therapeutic device 700 automatically. By way of example and not limitation, this may allow a user to remotely or manually move the wearable therapeutic device 700 along the transportation apparatus to different areas within or upon an article of clothing that may be proximate to different portions of the user's body to target different areas in need of therapeutic treatment.

In some aspects, the movement of the wearable therapeutic device 700 may be remotely facilitated via at least one computing device 720. By way of example and not limitation, a user may indicate an area on the body in need of therapeutic treatment via the computing device 720, and the wearable therapeutic device 700 maybe be configured to move to a position most advantageous to achieve the desired therapeutic effect. For example, a user may indicate via the computing device 720 that the user is experiencing neck pain. The computing device 720 may then transmit one or more instructions to the wearable therapeutic device 700 to cause the wearable therapeutic device 700 to move to one or more various locations proximate to the neck region to provide at least one therapeutic treatment thereto.

In some aspects, a user may use the computing device 720 to facilitate movement of the wearable therapeutic device 700 to different areas of the body via the transportation apparatus. In some aspects, a housing 710 of the wearable therapeutic device 700 may be configured to pivot, rotate, or move to facilitate different intensities, orientations, or directions of the light source and the light emitted therefrom. In some aspects, a user may facilitate this movement of the housing 710 via the computing device 720. By way of example and not limitation, this may allow the user to vary the treatment effects of the light source without moving the housing 710 from its location. In some aspects, this may allow a user to receive more precise treatment for one or more of a variety of ailments.

### CONCLUSION

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination or in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. A wearable therapeutic device, comprising:
at least one light source, wherein the at least one light source is configured to provide at least one therapeutic effect to a user;
at least one power source; wherein the at least one power source is configured to supply power to the at least one light source; and
at least one receptacle, wherein at least one receptacle is configured to at least partially enclose the at least one light source and the at least one power source, wherein the at least one receptacle is configured to at least temporarily secure the at least one light source and the at least one power source to one or more articles of clothing.

2. The wearable therapeutic device of claim 1, wherein the wearable therapeutic device further comprises at least one connection mechanism.

3. The wearable therapeutic device of claim 2, wherein the wearable therapeutic device further comprises at least one housing, wherein the at least one housing comprises the at least one light source, wherein the at least one connection mechanism is configured to removably connect the at least one housing to the at least one receptacle.

4. The wearable therapeutic device of claim 2, wherein the at least one connection mechanism is configured to electrically connect the at least one power source to the at least one light source.

5. The wearable therapeutic device of claim 1, wherein the wearable therapeutic device further comprises at least one circuit board.

6. The wearable therapeutic device of claim 5, wherein the wearable therapeutic device further comprises one or more electrical connections, wherein the one or more electrical connections facilitate at least one electrical connection between the at least one light source and the at least one power source via the at least one circuit board.

7. The wearable therapeutic device of claim 1, wherein the wearable therapeutic device further comprises at least one lens, wherein the at least one light source is configured to emit light through the at least one lens.

8. The wearable therapeutic device of claim 7, wherein the wearable therapeutic device further comprises at least one housing, wherein the at least one housing comprises the at least one light source and the at least one lens.

9. The wearable therapeutic device of claim 8, wherein the at least one receptacle comprises at least one transportation apparatus, and wherein the at least one transportation apparatus is configured to facilitate movement of the at least one housing within the at least one receptacle.

10. A system for providing a therapeutic effect, comprising:
at least one wearable therapeutic device comprising:
at least one light source, wherein the at least one light source is configured to provide at least one therapeutic effect to a user;
at least one power source; wherein the at least one power source is configured to supply power to the at least one light source;
at least one receptacle, wherein the at least one receptacle is configured to at least partially enclose the at least one light source and the at least one power source, wherein the at least one receptacle is configured to at least temporarily secure the at least one light source and the at least power source to one or more articles of clothing; and
one or more articles of clothing, wherein the at least one wearable therapeutic device is configured to be at least temporarily secured to at least one portion of each of the one or more articles of clothing.

11. The system for providing a therapeutic effect of claim 10, wherein the at least one wearable therapeutic device further comprises at least one circuit board.

12. The system for providing a therapeutic effect of claim 11, wherein the at least one wearable therapeutic device further comprises one or more electrical connections, wherein the one or more electrical connections facilitate at least one electrical connection between the at least one light source and the at least one power source via the at least one circuit board.

13. The system for providing a therapeutic effect of claim 10, wherein the at least one wearable therapeutic device further comprises at least one lens, wherein the at least one light source is configured to emit light through the at least one lens.

14. The system for providing a therapeutic effect of claim 12, wherein the at least one wearable therapeutic device further comprises a first at least one connection mechanism and a second at least one connection mechanism, wherein the first at least one connection mechanism, wherein the first at least one connection mechanism is connected to the at least one light source via the one or more electrical connections and the second at least one connection mechanism is connected to the at least one power source via the one or more electrical connections, wherein attaching the first at least one connection mechanism to the second at least one connection mechanism completes an electrical circuit between the at least one light source and the at least one power source.

15. The system for providing a therapeutic effect of claim 14, wherein the at least one wearable therapeutic device further comprises at least one housing, wherein the at least one housing comprises the at least one light source, wherein attaching the first at least one connection mechanism to the second at least one connection mechanism removably connects the housing to the at least one receptacle.

16. A wearable therapeutic device, comprising:
at least one light source, wherein the at least one light source is configured to provide at least one therapeutic effect to a user;
at least one power source, wherein the at least one power source is configured to supply power to the at least one light source;
at least one lens, wherein the at least one light source is configured to emit light through the at least one lens;
one or more electrical connections, wherein the one or more electrical connections facilitate at least one electrical connection between the at least one light source and the at least one power source via at least one circuit board;
at least one housing, wherein the at least one housing comprises the at least one light source and the at least one lens;
a first at least one connection mechanism, wherein the first at least one connection mechanism is connected to the at least one light source via the one or more electrical connections;
a second at least one connection mechanism, wherein the second at least one connection mechanism is connected to the at least one power source via the one or more electrical connections, wherein attaching the first at least one connection mechanism to the second at least one connection mechanism completes an electrical circuit between the at least one light source and the at least one power source; and
at least one receptacle, wherein the at least one receptacle is configured to at least temporarily secure the at least one housing to one or more articles of clothing, wherein attaching the first at least one connection mechanism to the second at least one connection mechanism removably connects the at least one housing to the at least one receptacle.

17. The wearable therapeutic device of claim 16, wherein the wearable therapeutic device further comprises at least one sensor, wherein the at least one sensor is configured to detect at least one datum related to the use of the wearable therapeutic device.

18. The wearable therapeutic device of claim 17, wherein the wearable therapeutic device further comprises at least one transmitting device.

19. The wearable therapeutic device of claim 18, wherein the wearable therapeutic device further comprises at least one storage medium.

20. The wearable therapeutic device of claim 16, wherein the at least one receptacle comprises at least one transportation apparatus, and wherein the at least one transportation apparatus is configured to facilitate movement of the at least one housing within the at least one receptacle.
